(19) United States
(12) Patent Application Publication
(10) Pub. No.: US 2011/0230449 A1
(43) Pub. Date: Sep. 22, 2011

(54) Alkoxylated Phosphoric Acid Triesters With A High Degree Of Alkoxylation

(75) Inventors: Peter Klug, Grossostheim (DE); Franz-Xaver Scherl, Burgkirchen (DE); Waltraud Simsch, Kelkheim (DE); Adelgunde Oberhauser, Neuoetting (DE)

(73) Assignee: CLARIANT FINANCE (BVI) LIMITED, Tortola (VG)

(21) Appl. No.: 12/671,802

(22) PCT Filed: Jul. 29, 2008
(86) PCT No.: PCT/EP2008/006221

§ 371 Date: Jul. 29, 2010

(30) Foreign Application Priority Data
Aug. 2, 2007 (DE) 10 2007 036 187.6
Jul. 29, 2008 (EP) PCT/EP2008/006221

(51) Int. Cl. A61K 8/55; C07F 9/113; A61P 19/00

(52) U.S. Cl. 514/129; 558/186

(57) ABSTRACT

The invention relates to alkoxylated phosphoric acid triesters of formula (I) wherein x, y and z respectively stand for a number between 51 and 200. The alkoxylated phosphoric acid triesters of formula (I) are characterised especially by advantageous thickening properties.

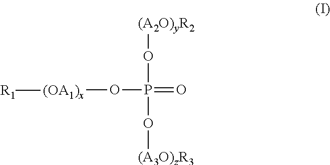

The invention relates to phosphoric triesters which derive from alkoxylated fatty alcohols having more than 50 alkoxy groups.

Phosphoric esters are unobjectionable from the standpoints of toxicology and ecotoxicology, are skin-kind on account of their neutral pH levels, and are highly suitable for use in cosmetic formulations.

Alkyl- and alkenylphosphoric esters are typically prepared by condensing fatty alcohols with diphosphorus pentoxide or orthophosphoric acid, giving mixtures of mono-/di-/triester, with a major fraction of monoester and diester.

JP 09-268193 describes a method of preparing phosphoric triesters by reacting phosphorus oxychloride with a fatty alcohol or an alkoxylated fatty alcohol in the presence of a catalyst selected from TiCl₄, MgCl₂ or AlCl₃. Phosphoric triesters are obtained which may carry up to 50 —CH₂CH₂O units (EO). These ethoxylated phosphoric esters can be used with advantage as thickeners. Disadvantages, however, are their low water solubility and the difficulty of processing them to aqueous formulations.

Moreover, phosphoric triesters are desired which have no chlorine-containing impurities as a result of their preparation.

The object was therefore to provide phosphoric triesters which not only have good thickening properties but at the same time can be readily incorporated into compositions on an aqueous basis, and have good compatibility with cosmetic ingredients.

It has surprisingly been found that this object is achieved by phosphoric triesters having a degree of alkoxylation >50. These triesters are notable for a good thickening action in aqueous compositions, are highly water-soluble, and can be easily processed to aqueous formulations.

The present invention accordingly provides phosphoric triesters of the formula (I)

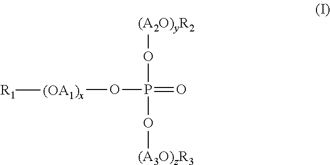

in which
R₁, R₂, and R₃ may be alike or different and are a linear or branched, saturated alkyl group having 6 to 30, preferably 8 to 22, and more preferably 12 to 18 carbon atoms, a linear or branched, mono- or polyunsaturated alkenyl group having 6 to 30, preferably 8 to 22, and more preferably 12 to 18 carbon atoms, or an aryl group, more particularly a phenyl group, which may be substituted by 1 to 3 branched alkyl groups each independently of one another containing 3 to 18 and preferably 4 to 12 carbon atoms, the individual groups (OA₁)ₓ, (A₂O)_{y}, and (A₃O)_{z} each independently of one another are composed of units selected from CH₂CH₂O, C₃H₆O and C₄H₈O, it being possible for the units CH₂CH₂O, C₃H₆O and C₄H₈O within the individual groups (OA₁)ₓ, (A₂O)_{y}, and (A₃O)_{z} to be in a blockwise or randomly distributed arrangement, and
x, y and z each independently of one another are a number from 51 to 200, preferably from 55 to 150, and more preferably from 60 to 100.

Preferably the radicals R₁, R₂, and R₃ of the phosphoric esters of the formula (I), which may be alike or different, are a linear or branched, saturated alkyl group having 6 to 30, preferably 8 to 22 and more preferably 12 to 18 carbon atoms, or are a linear or branched, mono- or polyunsaturated alkenyl group having 6 to 30, preferably 8 to 22, and more preferably 12 to 18 carbon atoms.

With further preference the units OA₁, OA₂, and OA₃ in the phosphoric esters of the formula (I) are CH₂CH₂O.

With particular preference, the radicals R₁—(OA₁))ₓ, R₂—(OA₂)_{y}, and R₃—(OA₃)_{z} in the phosphoric esters of the formula (I) are derived from fatty alcohol ethoxylates selected from fatty alcohol ethoxylates having 51 to 200 EO units (EO=CH₂CH₂O), preferably having 55 to 150 EO units, and more preferably having 60 to 100 EO units, the respective fatty alcohol residue R₁₀—, R₂₀—, and R₃₀— being derived from alcohols selected from octanol, decanol, dodecanol, tetradecanol, hexadecanol, octadecanol, elcosanol, behenyl alcohol, fatty alcohols having C-chain cuts between 8 and 22, preferably C₁₀/C₁₂ fatty alcohol, C₁₂/C₁₄ fatty alcohol, C₁₂/C₁₆ fatty alcohol, and C₁₆/C₁₈ fatty alcohol, more preferably C_{16/18} fatty alcohol ethoxylate having 80 ethylene oxide units (e.g., Genapol® T 800), branched fatty alcohols, preferably Guerbet alcohols, and monounsaturated fatty alcohols, preferably delta-9-cis-hexadecanol, delta-9-cis-octadecanol, trans-9-octadecanol, and cis-delta-11-octadecanol.

With further preference the groups (OA₁)ₓ, (OA₂)_{y}, and (OA₃)_{z} in the phosphoric triesters of the formula (I) are each constructed of CH₂CH₂O and C₃H₆O units, it being possible for the CH₂CH₂O and C₃H₆O units within the individual groups (OA₁)ₓ, (OA₂)_{y}, and (OA₃)_{z} to have a blockwise or randomly distributed arrangement, and each contain 51 to 199, preferably 55 to 150, and more preferably 60 to 100 CH₂CH₂O units and 1 to 20, preferably 1 to 10, and more preferably 2 to 5 C₃H₆O units.

The phosphoric triesters of the formula (I) can preferably be prepared by reacting phosphoric acid or phosphoric acid derivatives with alkoxylated fatty alcohols, the alkoxylated fatty alcohols carrying at least 51 alkoxy groups.

The phosphoric esters of the formula (I) are prepared more preferably by reacting phosphoric acid or a phosphoric acid derivative selected from orthophosphoric acid, tetraphosphorus decaoxide, polyphosphoric acid, phosphorus oxychloride or phosphorus pentachloride with fatty alcohol alkoxylates at temperatures between 150 and 250° C., preferably between 180 and 240° C. and with particular preference between 200 and 230° C.

The phosphoric esters of the formula (I) are prepared with particular preference by reacting orthophosphoric acid, polyphosphoric acid or tetraphosphorus decaoxide, and very preferably by reacting orthophosphoric acid, with fatty alcohol alkoxylates. This embodiment of the process produces phosphoric triesters of the formula (I) which are chlorine-free. This means in particular that they do not contain any chlorine impurities.

In another preferred embodiment of the present invention the phosphoric esters of the formula (I) are chlorine-free.

The phosphoric triesters of the formula (I) may advantageously also be present together with other phosphoric esters in mixtures.

Further provided by the present invention, accordingly, are mixtures comprising one or more phosphoric triesters of the formula (I) and one or more phosphoric esters of the formula (II)

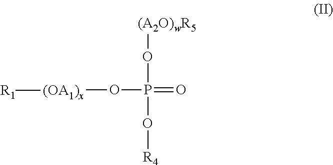

in which
R₁ is a linear or branched, saturated alkyl group having 6 to 30, preferably 8 to 22, and more preferably 12 to 18 carbon atoms, a linear or branched, mono- or polyunsaturated alkenyl group having 6 to 30, preferably 8 to 22, and more preferably 12 to 18 carbon atoms, or an aryl group, more particularly a phenyl group, which may be substituted by 1 to 3 branched alkyl groups, each independently of one another containing 3 to 18 and preferably 4 to 12 carbon atoms,
R₄ is H, Li⁺, Na⁺, K⁺, Mg⁺⁺, Ca⁺⁺, Al⁺⁺⁺, NH₄⁺ or quaternary ammonium ions [HNR^{a}R^{b}R^{c}]⁺, in which R^{a}, R^{b}, and R^{c} independently of one another are hydrogen, a linear or branched alkyl group having 1 to 22 carbon atoms, a linear or branched, mono- or polyunsaturated alkenyl group having 2 to 22 carbon atoms, a linear mono-hydroxyalkyl group having 2 to 10 carbon atoms, preferably a mono-hydroxyethyl or mono-hydroxypropyl group, or a linear or branched di-hydroxyalkyl group having 3 to 10 carbon atoms,
R₅ possesses the definition of R₁ or R₄,
the individual groups (OA₁)ₓ and (A₂O)_{w} each independently of one another are composed of units selected from CH₂CH₂O, C₃H₆O and C₄H₈O, it being possible for the units CH₂CH₂O, C₃H₆O and C₄H₈O within the individual groups (OA₁)ₓ and (A₂O)_{w} to be in a blockwise or randomly distributed arrangement,
x is a number from 51 to 200, preferably from 55 to 150, and more preferably from 60 to 100, and
w is 0 or a number from 51 to 200, preferably from 55 to 150, and more preferably from 60 to 100.

Preferred mixtures of the invention are composed of the compounds of the formula (I) and (II).

In the mixtures of the invention comprising phosphoric esters of the formula (I) and (II), the amount of the phosphoric triesters of formula (I) is preferably greater than 80.0%, more preferably from 82.0% to 95.0%, and with particular preference from 85.0% to 89.0%, by weight, based on the total weight of the phosphoric esters of formula (I) and formula (II).

The degree of neutralization of the unesterified phosphorus valences (P—OH) in the phosphoric esters of formula (II) may be between 0% and 100%.

The remaining free valences on the phosphorus atom may be acid groups, or else counterions, selected from Li⁺, Na⁺, K⁺, Mg⁺⁺, Ca⁺⁺, Al⁺⁺⁺, NH₄⁺, quaternary ammonium ions [HNR²R^{b}R^{c}]⁺, in which R², R^{b}, and R^{c} independently of one another are hydrogen, a linear or branched alkyl group having 1 to 22 carbon atoms, a linear or branched, mono- or polyunsaturated alkenyl group having 2 to 22 carbon atoms, a linear mono-hydroxyalkyl group having 2 to 10 carbon atoms, preferably a mono-hydroxyethyl or mono-hydroxypropyl group, or a linear or branched di-hydroxyalkyl group having 3 to 10 carbon atoms.

In another preferred embodiment of the invention the phosphoric esters of the formula (II) are neutralized, with a degree of neutralization of 0%-20.0%.

In a further preferred embodiment of the invention the phosphoric esters of the formula (II) are neutralized, with a degree of neutralization of 20.1%-100%.

The mixtures of the invention may comprise one or more phosphoric esters of the formula (II) in which R⁵ possesses the definition of R⁴, and w is 0. These compounds are mono-phosphoric esters. In the mixtures of the invention they are present preferably in an amount <3.0%, more preferably <1.0%, and with particular preference <0.1%, by weight, based on the total weight of the phosphoric esters of formula (I) and (II). In the mono-phosphoric esters of the formula (II), R₄ and R₅ may be alike or different.

In another preferred embodiment of the invention, the mixtures of the invention comprise one or more phosphoric esters of the formula (II) in which R₅ possesses the definition of R₁, and w is a number from 51 to 200, preferably from 55 to 150, and more preferably from 60 to 100. These compounds are di-phosphoric esters. In the mixtures of the invention they are present preferably in an amount from 5.0% to 18.0%, more preferably from 10.0% to 17.0%, and with particular preference from 11.0% to 15.0%, by weight, based on the total weight of the phosphoric esters of formula (I) and (II). In the di-phosphoric esters of the formula (II), the radicals R₁ and R₅ may be alike or different.

Mixtures of the phosphoric esters of the formulae (I) and (II) are prepared preferably by reacting phosphoric acid or a phosphoric acid derivative selected from orthophosphoric acid, tetraphosphorus decaoxide, and polyphosphoric acid, and more preferably by reacting orthophosphoric acid, with fatty alcohol alkoxylates at temperatures between 150 and 250° C., preferably between 180 and 240° C., and with particular preference between 200 and 230° C.

This process produces mixtures of the phosphoric esters of the formulae (I) and (II) which are chlorine-free. This means in particular that these phosphoric esters of the formulae (I) and (II) contain no chlorine impurities.

Further-preferred mixtures of the invention comprising phosphoric esters of formula (I) and formula (II) are chlorine-free.

The following examples and applications are intended to elucidate the invention in more detail, but without restricting it to them. All percentages are weight % (% by weight).

### PREPARATION EXAMPLES

### General Operational Instructions

In the preparation of the phosphoric esters of the invention, phosphoric acid (85% strength) and fatty alcohol ethoxylate are used in a defined molar ratio. For this purpose, all of the reactants are charged to a stirred apparatus with heating mantle, water separator with condenser, and vacuum connection. The mixture is heated to 100° C., evacuated three times to 100 mbar, and then ventilated again with nitrogen. After a further 4 hours of inertizing (introduction of nitrogen at 20 liters/hour) at 100° C., the batch is heated to 230° C., with introduction of nitrogen, and esterified (water discharge). The reaction times are 24 to 42 hours (reckoned from an esterification temperature of 230° C.), more particularly 40 hours. The residual acid number at that point is <3 mg KOH/g. This corresponds to a conversion of approximately 93% to 96% (based on initial acid number). After the end of reaction, the product is cooled to 80° C. and poured out into a tray, and the solidified melt is comminuted.

### Example 1

Ester formed from 11.4 g of phosphoric acid and 935.1 g of ceteareth-80 (C_{16/18} fatty alcohol+80 mol of ethylene oxide) in a molar ratio of 1:3, residual acid number: 0.8 mg KOH/g (96% conversion), ³¹P-NMR: diester/triester=15/85 mol %. The ester is a white wax having a melting point of around 40° C.

1. A phosphoric acid triester of the formula (I) 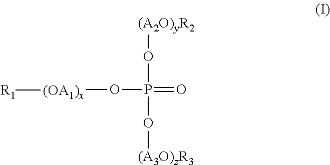
wherein
R₁, R₂, and R₃ may be alike or different and are a linear or branched, saturated alkyl group having 6 to 30 carbon atoms, a linear or branched, mono- or polyunsaturated alkenyl group having 6 to 30 carbon atoms, or an aryl group, which may be substituted by 1 to 3 branched alkyl groups each independently of one another containing 3 to 18 carbon atoms,
the individual groups (OA₁)ₓ, (A₂O)_{y}, and (A₃O)_{z} each independently of one another are composed of units selected from the group consisting of CH₂CH₂O, C₃H₆O and C₄H₈O, it being possible for the units CH₂CH₂O, C₃H₆O and C₄H₈O within the individual groups (OA₁)ₓ, (A₂O)_{y}, and (A₃O)_{z} to be in a blockwise or randomly distributed arrangement, and
x, y and z each independently of one another are a number from 51 to 200.

2. A phosphoric acid triester as claimed in claim 1, wherein the radicals R₁, R₂, and R₃ may be alike or different and are a linear or branched, saturated alkyl group having 6 to 30 carbon atoms, or are a linear or branched, mono- or polyunsaturated alkenyl group having 6 to 30 carbon atoms.

3. A phosphoric acid triester as claimed in claim 1, wherein the units OA₁, OA₂, and OA₃ are CH₂CH₂O.

4. A phosphoric acid triester as claimed in claim 1, wherein the radicals R₁, R₂, and R₃ may be alike or different and are a linear or branched, saturated alkyl group having 12 to 30 carbon atoms, or are a linear or branched, mono- or polyunsaturated alkenyl group having 12 to 30 carbon atoms, and the units OA₁, OA₂, and OA₃ are CH₂CH₂O.

5. A phosphoric acid triester as claimed in claim 1, wherein the radicals R₁—(OA₁)ₓ, R₂—(OA₂)_{y}, and R₃—(OA₃)_{z} are derived from fatty alcohol ethoxylates selected from fatty alcohol ethoxylates having 51 to 200 EO units (EO=CH₂CH₂O), the respective fatty alcohol residue R₁O—, R₂O—, and R₃O— being derived from alcohols selected from the group consisting of octanol, decanol, dodecanol, tetradecanol, hexadecanol, octadecanol, eicosanol, behenyl alcohol, fatty alcohols having C-chain cuts between 8 and 22, branched fatty alcohols, and monounsaturated fatty alcohols.

6. A phosphoric acid triester as claimed in claim 1, wherein the groups (OA₁)ₓ, (OA₂)_{y}, and (OA₃)_{z} are each constructed of CH₂CH₂O and C₃H₆O units, it being possible for the CH₂CH₂O and C₃H₆O units within the individual groups (OA₁)ₓ, (OA₂)_{y}, and (OA₃)_{z} to have a blockwise or randomly distributed arrangement, and each contain 51 to 199 CH₂CH₂O units and 1 to 20 C₃H₆O units.

7. A phosphoric acid triester as claimed in claim 1, which is chlorine-free.

8. A mixture comprising at least one phosphoric acid triester of the formula (I) as claimed in claim 1, and at least one phosphoric acid ester of the formula (II) 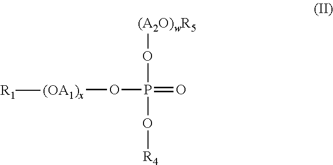
wherein
R₁ is a linear or branched, saturated alkyl group having 6 to 30 carbon atoms, a linear or branched, mono- or polyunsaturated alkenyl group having 6 to 30 carbon atoms, or an aryl group, which may be substituted by 1 to 3 branched alkyl groups, each independently of one another containing 3 to 18 carbon atoms,
R₄ is H, Li⁺, Na⁺, K⁺, Mg⁺⁺, Ca⁺⁺, Al⁺⁺⁺, NH₄⁺ or quaternary ammonium ions [HNR^{a}R^{b}R^{c}]⁺, wherein R^{a}, R^{b}, and R^{c} independently of one another are hydrogen, a linear or branched alkyl group having 1 to 22 carbon atoms, a linear or branched, mono- or polyunsaturated alkenyl group having 2 to 22 carbon atoms, a linear mono-hydroxyalkyl group having 2 to 10 carbon atoms, or a linear or branched di-hydroxyalkyl group having 3 to 10 carbon atoms,
R₅ is defined as R₁ or R₄,
the individual groups (OA₁)ₓ and (A₂O)_{w} each independently of one another are composed of units selected from the group consisting of CH₂CH₂O, C₃H₆O and C₄H₈O, it being possible for the units CH₂CH₂O, C₃H₆O and C₄H₈O within the individual groups (OA₁)ₓ and (A₂O)_{w} to be in a blockwise or randomly distributed arrangement,
x is a number from 51 to 200, and
w is 0 or a number from 51 to 200.

9. A mixture as claimed in claim 8, consisting of the compounds of the formula (I) and (II).

10. A mixture as claimed in claim 8, wherein the amount of the at least one phosphoric acid triester of formula (I) is greater than 80.0% by weight, based on the total weight of the phosphoric esters of formula (I) and formula (II).

11. A mixture as claimed in claim 8, which comprises at least one phosphoric ester of the formula (II), in which R₅ is defined as R₁ and w is a number from 51 to 200, wherein the at least one phosphoric acid ester of formula (II) is present in an amount from 5.0% to 18.0% by weight, based on the total weight of the phosphoric esters of formula (I) and (II).

12. A mixture as claimed in claim 8, which is chlorine-free.
